(19) United States
(12) Plant Patent Application Publication
(10) Pub. No.: US 2003/0079260 P1
(43) Pub. Date: Apr. 24, 2003

(54) Hybrid Tea rose plant named 'Meileeuw'

(76) Inventor: Alain A. Meilland, Antibes (FR)

Correspondence Address: Benton S. Duffett, Jr., BURNS, DOANE, SWECKER & MATHIS, L.L.P., P.O. Box 1404, Alexandria, VA, 22313-1404, US

(21) Appl. No.: 10/306,411

(22) Filed: Nov. 29, 2002

Related U.S. Application Data
(63) Continuation of application No. 09/698,237, filed on Oct. 30, 2000, now abandoned.

(51) Int. Cl. A01H 5/00

(52) U.S. Cl. PLT/131

(57) ABSTRACT

A new and distinct variety of Hybrid Tea rose plant is provided which forms attractive double blossoms that are red on the upper surface and yellow-orange suffused with pinkish-red on the under surface. The plant exhibits an erect strong vigorous growth habit, dense semi-glossy medium green foliage, and good disease resistance. The attractive medium green foliage contrasts nicely with the blossom coloration. The new variety is particularly well suited for cut flower production in the greenhouse.

### SUMMARY OF THE INVENTION

The new Hybrid Tea variety of Rosa hybrida of the present invention was discovered during 1997 at Le Cannet des Maures, Var, France while growing among plants of the ’Meicofum’ variety (U.S. Plant Pat. No. 9,997). The new variety is believed to be the result of a natural mutation of unknown causation. The new variety was selected and was carefully preserved primarily because of its distinctive blossom coloration. Had this mutation not been discovered and preserved it would have been lost to mankind.

It was found that the new variety of the present invention

(a) exhibits an erect growth habit,

(b) exhibits a strong vigorous growth habit,

(c) forms attractive double blossoms that are red on the upper surface and yellow-orange suffused with pinkish-red on the under surface,

(d) forms dense medium green semi-glossy foliage that contrasts nicely with the blossom coloration, and

(e). is particularly well suited for growing for cut flower production in the greenhouse.

The disease resistance of the new variety is good.

The new variety well meets the needs of the horticultural industry and can be used to advantage for forming distinctive cut flowers indoors.

The new variety of the present invention can be readily distinguished from the parent ’Meicofum’ variety which forms bicolored blossoms that are reddish-orange on the upper surface and a lighter creamy yellow on the under surface. The blossoms of the present variety are red on the upper surface and yellow-orange suffused with pinkish-red on the under surface.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ’Meileeuw’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during November while budded on Rosa indica understock and growing in greenhouses at Le Cannet des Maures, Var, France. Dimensions in centimeters are indicated at the bottom of the photograph.

FIG. 1—illustrates a specimen of a young shoot; FIG. 2—illustrates a specimen of a floral bud before the opening of the sepals; FIG. 3—illustrates a specimen of a floral bud at the opening of the sepals; FIG. 4—illustrates a specimen of a floral bud at the opening of the petals; FIG. 5—illustrates a specimen of a flower in the course of opening; FIG. 6—illustrates a specimen of an open flower—plan view obverse; FIG. 7—illustrates a specimen of an open flower—plan view reverse; FIG. 8—illustrates a specimen of a fully open flower—plan view—obverse; FIG. 9—illustrates a specimen of a fully open flower—plan view—reverse; FIG. 10—illustrates a specimen of a floral receptacle showing the arrangement of the stamens and pistils; FIG. 11—illustrates a specimen of a floral receptacle showing the arrangement of the pistils (stamens removed); FIG. 12—illustrates a specimen of a flowering stem;

-4FIG. 13—illustrates a specimen of a main branch;

FIG. 14—illustrates a specimen of a leaf with three leaflets plan view—upper surface;

FIG. 15—illustrates a specimen of a leaf with five leaflets plan view—under surface; and

FIG. 16—illustrates a specimen of a leaf with seven leaflets plan view—upper surface.

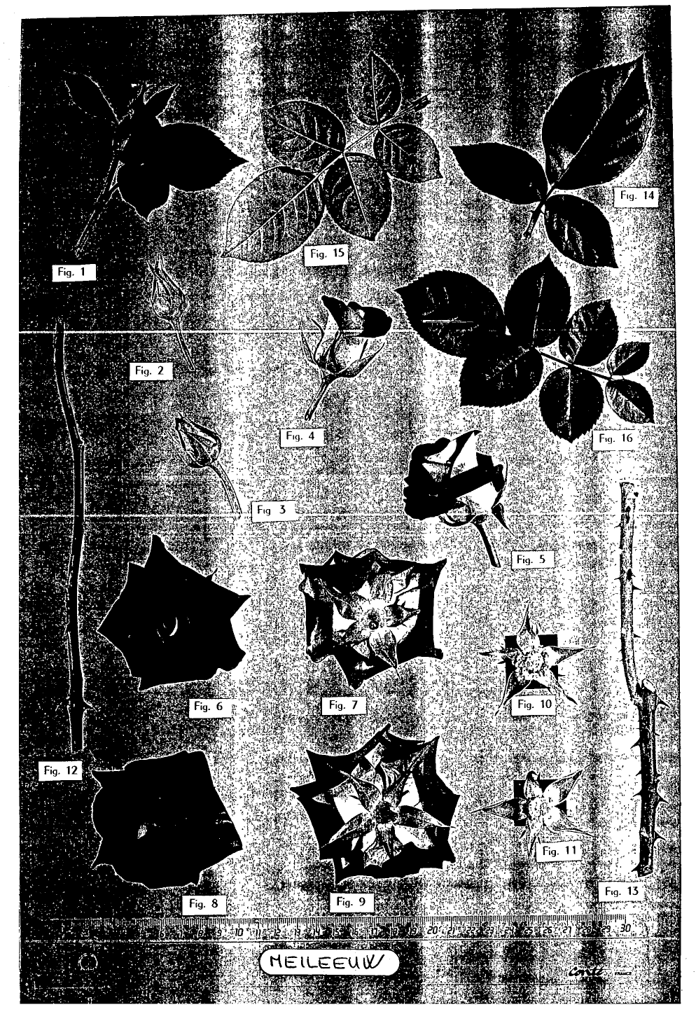

### DETAILED DESCRIPTION

The chart used in the identification of the colors is that of The Royal Horticultural Society (R.H.S. Colour Chart). The description is based on the observation of two year-old plants during November while budded on Rosa indica understock and growing in greenhouses at Le Cannet des Maures, Var, France.

Class: Hybrid Tea.

Plant:

height.—when pruned to a height of 85 cm, floral stems having a length of approximately 50 to 70 cm commonly are produced at the end of the growing season.

habit.—erect.

Branches:

color.—young stems: near Yellow-Green Group 147B. adult wood: near Green Group 137D.

thorns.—size: medium (as illustrated). quantity: moderately numerous (as illustrated). color: near Yellow-Green Group 144C on young stems and adult wood.

Leaves:

stipules.—adnate, pectinate, and rather narrow.

petioles.—upper surface: near Green Group 137C and non-glandular. under surface: near Green Group 138B and bear a few prickles.

leaflets.—number: 3, 5 (most often), and 7. shape: elliptical with symmetrical tips and obtuse base. serration: simple and regular (as illustrated). texture: consistent. general appearance: very dense, medium green, and semi-glossy. color (young foliage): upper surface: near Yellow-Green Group 147A and commonly bordered with reddish coloration. under surface: near Yellow-Green Group 147B. color (adult foliage): upper surface: near Yellow-Green Group 147A. under surface: near Yellow-Green Group 147B.

Inflorescence

number of flowers.—usually one per stem.

peduncle.—broad and rigid, near Yellow-Green Group 146A, commonly with prickles, and the length is approximately 10 cm on average.

sepals.—upper surface: tomentose and near Yellow-Green Group 147B and 147C in coloration. under surface: near Yellow-Green Group 146B and 146C commonly with some light reddish coloration and commonly with a few small extensions.

buds.—shape: conical. size: fairly large. length: approximately 3 cm on average.

flower.—shape: cup-shaped. diameter: approximately 11 to 12 cm on average. color (when opening begins): upper surface: near Red Group 45A and lightly suffused with Currant Red, Red Group 46A. under surface: near Yellow Group 13C to Yellow-Orange Group 14B and suffused with Rhodonite Red, Red Group 51B. color (when blooming): upper surface: near Red Group 45A and lightly suffused with Currant Red, Red Group 46A. under surface: near Yellow Group 12C and Yellow Group 12D suffused with Orient Pink, Red Group 36D and Neyron Rose, Red Group 55C. color (at end of opening): upper surface: near Red Group 45A and lightly suffused with Currant Red, Red Group 46A. under surface: near Yellow Group 12C and Yellow Group 12D suffused with Red Group 36D and Red Group 55C. fragrance: none. lasting quality: long, the blossoms commonly last approximately 10 to 12 days on the plant and approximately 10 to 12 days when cut and placed in a vase. petal shape: rounded with reflexed edges. stamen number: approximately 95 on average. anthers: near Yellow Group 13B in coloration. filaments: near Yellow Group 12B in coloration. pistils: approximately 112 on average. stigmas: near Orange-Red Group 34C in coloration. receptacle: near Yellow-Green Group 146A in coloration, and funnel-shaped in longitudinal section.

Development

vegetation.—vigorous and strong.

blooming.—typical.

resistance to diseases.—good.

I claim:

1. A new and distinct variety of Hybrid Tea rose plant characterized by the following combination of characteristics:
(a) exhibits an erect growth habit,
(b) exhibits a strong vigorous growth habit,
(c) forms attractive double blossoms that are red on the upper surface and yellow-orange suffused with pinkish-red on the under surface,
(d) forms dense medium green semi-glossy foliage that contrasts well with the blossom coloration, and
(e) is particularly well suited for growing for cut flower production in the greenhouse; substantially as herein shown and described.
